(19) United States
(12) Patent Application Publication
(10) Pub. No.: US 2007/0190126 A1
(43) Pub. Date: Aug. 16, 2007

(54) Method for treating generalized and focal peripheral neuropathies

(76) Inventor: Daniel M. Stuart, Coronado, CA (US)

Correspondence Address: DLA PIPER US LLP, 4365 EXECUTIVE DRIVE, SUITE 1100, SAN DIEGO, CA, 92121-2133, US

(21) Appl. No.: 11/705,651

(22) Filed: Feb. 12, 2007

Related U.S. Application Data
(60) Provisional application No. 60/773,196, filed on Feb. 14, 2006.

(51) Int. Cl. A61K 31/714; A61K 9/70

(52) U.S. Cl. 424/449; 514/52

(57) ABSTRACT

A composition providing cobalamine, cobalamine subtype or cobalamine analog as an effective agent for topical or transdermal administration to alleviate pain associated with a generalized or focal peripheral neuropathic condition, such as carpal tunnel syndrome.

### FIELD OF THE INVENTION

The invention relates to methods and compositions for alleviating pain associated with peripheral neuropathies.

### BACKGROUND OF THE INVENTION

Peripheral neuropathies are often characterized by inflammation and swelling in tissues adjacent to the affected nerves, often accompanied by pain, tingling or numbness at sites traversed or innervated thereby. Peripheral neuropathies may occur in conjunction with disease or conditions that stress the body and nervous system, such as alcoholism, diabetes, arthritis and pregnancy (generalized peripheral neuropathies). They may also result from direct impact on a nerve passing through or relatively close to a joint or bony prominence, usually near the skin (focal peripheral neuropathies, including compression neuropathies).

A common example of the latter condition occurs in compression neuropathies, such as carpal tunnel syndrome (CTS), affecting primarily the median nerve and wrist; cubital tunnel syndrome, affecting primarily the ulnar nerve and elbow; and tarsel tunnel syndrome, affecting primarily the tibial nerve and lower leg. All of these conditions involve nerves which, together with tendons and ligaments, pass through a space limited area, such as the area surrounding a joint.

For example, CTS involves compression of the median nerve of the hand, which passes through a “tunnel” defined by the bones of the wrist and a ligament. Swelling of the tendons in the area in response to injury or repetitive stresses compresses the median nerve. Pain in the hand and wrist results, which may extend to the forearm, elbow and shoulder.

Conventional treatment for CTS involves providing mechanical support to the affected area, such as a splint, and/or injection of a corticosteroid. The latter is painful, and the former rarely resolves the condition completely. Orally administered non-steroidal anti-inflammatories, diuretics and vitamin B-6 can help to relieve symptoms as well, albeit temporarily and at the risk of systemic side effects. Surgery may also be an option for persistently symptomatic suffers.

With fewer risks of systemic side effects and less painful application, topical treatment for peripheral neuropathies is an attractive alternative to conventional therapy. To date, however, topical balms and liniments have enjoyed limited success in treating such conditions, because of limited efficacy, inability of the active drug to cross the dermal barrier, or both. Therefore, orally administered drugs, steroid injections and splinting remain the predominantly used methods for non-surgical treatment of CTS (see, e.g., the review paper by O'Connor, et al., Cochrane Database Syst. Rev., CD003219, 2003).

### SUMMARY OF THE INVENTION

The invention provides effective topical and transdermal compositions for use in alleviating pain associated with peripheral neuropathies, and methods for their use. In particular, the invention provides a pharmaceutically acceptable composition in which a cobalamine or cobalamine subtype serves as an effective active agent for relieving pain associated with the neuropathy. Although other actives may be present in the composition (e.g., to help reduce inflammation), only cobalamine or a cobalamine subtype need be present to provide the therapeutic benefit sought in the invention.

In a preferred embodiment of the invention, the active is cobalamine (vitamin B-12).

In a further embodiment of the invention, the cobalamine active is co-administered or admixed with an anti-inflammatory compound. Such compounds include vitamin B-1, B-2, B-3, B-6, B-9, alpha-lipoic acid, the alpha or gamma linoleic and linolenic acids, and the amino acid derivative acetyl-1-camitine, all of which have been observed to relieve pain by decreasing nerve inflammation in other contexts (see, e.g., Folkers, et al., PNAS USA, 81(22):7076-8, 1984; Wang, et al., Pain, 1116 (1-2):168, 2005; Yxfeldt, et al., Sand J Rheumatol., 32(4):205-10, 2003; Colker, et al,. Nutrition, 18(5):388-92, 2002; Haupt, al., Int. J. Clin. Pharmacol. Ther., 43(2):71-7, 2005; Medina-Santillan, et al., Proc. West. Pharmacol. Soc., 47:95-9, 2004; Franca, et.al., Eur. J. Pharmacol., 421(3):157-64, 2001; Granados-Soto, et al., Eur. J. Pharmacol., 492(1):35-40, 2004; Chiang, et al., Arthritis Rest Her., 7(6):R1254-62, 2005; Chiang, Am J. Med., 114(4):283-7, 2003; Sato, J. Neurol Sci., 15;23(1-2):13-8, 2005; Li, Zhonghua New Ke Za Zhi, 38(1):14-7, 1999; Yaqub, Clin Neurol Neurosurg., 94(2):105-11, 1992; and, Kuwabara, Intern Med, 38(6):472-5, 1999).

In particular, the linoleic and linolenic acids are precursors to prostaglandins, and have anti-inflammatory effects when applied according to the invention. Alpha linoleic and linolenic acids metabolize to the longer chain fatty acids eicosapentaenoic acid (EPA) and docosahexaenoic acid (DHA), and have been observed to exert a neuroprotective effect in certain in vivo models; e.g., of epilepsy. Alpha lipoic acid and camitine are antioxidants that have been used outside of the United States to treat conditions including nerve damage.

In another embodiment of the invention, the afore-mentioned pain reducers are administered alone, to treat pain in between or during other therapy.

Most preferably, the cobalamine active composition (with or without anti-inflammatory additives) is provided in a composition for topical application, as in a balm, liniment, lotion or gel form. A further preferred embodiment of the invention provides a cobalamine active composition for transdermal delivery. The cobalamine composition may also be administered topically or transdermally over a prolonged period, as by use of a patch and/or in an extended release form.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as illustrating practice of the invention, and do not limit its scope in any way.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Whenever used, any percentage is weight by weight (w/w) unless otherwise indicated.

A. Definitions.

As used herein, “active” refers to a pharmaceutically active compound or ingredient of a composition. “Cobalamine active” and “cobalamine composition” encompass cobalamine, cobalamine subtypes and cobalamine analogs, as defined below, unless context otherwise indicates.

As used herein, “cobalamine” is a cobalt-containing coordination compound produced principally by intestinal micro-organisms, also known as vitamin B12. Pharmaceutical grade cobalamine is commercially available, and preferred for use in the invention.

As used herein, “cobalamine subtypes” means mecobalamine, nitritcobalamin, sulphitocobalamin, aquacobalamin, hydroxocobalamin, cobamamide and the predominant coenzyme forms of methyl-cobalamine and adnosyl-cobalimin, together with other cobalamine variants having in vivo activity and bioavailability equivalent to, or better than, cobalamine. Pharmaceutical grade cobalamine subtype compounds are commercially available, and preferred for use in the invention.

“Pain reducers” means any compound with demonstrable pain-reduction activity when applied topically and includes, without limitation, vitamin B-1, B-2, B-3, B-6, B-9, alpha-lipoic acid, the alpha or gamma linoleic and linolenic acids, and the amino acid derivative acetyl-1-camitine.

As used herein, “topical delivery” means directly laying on or spreading on outer skin, e.g., by use of the hands or an applicator such as a wipe, puff, roller, or spray. Compositions of the invention useful in this context are said to be “topically deliverable.”

As used herein, “transdermal delivery” means to apply using mechanical or chemical means to ensure deeper or extended penetration of the active, such as by a transdermal patch, iontophoresis or sono-phonophoresis. Compositions of the invention useful in this context are said to be “transdermally deliverable.”

As used herein, “pharmaceutically acceptable” means that the compound(s) or composition(s) which the term describes are suitable for use in contact with tissues (e.g., the skin) without undue toxicity, incompatibility, instability, irritation, allergic response, and the like. An “effective amount” means an amount of compound(s) or composition(s) sufficient to alleviate pain associated with the peripheral neuropathy to be treated, but low enough to avoid serious side effects.

B. Cobalamine Active Compositions of the Invention.

The topical compositions useful in the present invention involve formulations suitable for topical application to skin, and will generally comprise a cobalamine active and a pharmaceutically acceptable topical carrier. Pain reducers may also be included in the topical cobalamine composition, or can be administered separately.

Pharmaceutically acceptable carriers preferred for use with the cobalamine actives and compositions of the invention may include sterile aqueous of non-aqueous solutions, suspensions, and emulsions. For example, suitable carriers for use in the invention include gels, glosses, creams, liquid carriers, thin liquid carriers, thick liquid carriers, waxes, oils, oil based carriers, water based carriers, serums, suspensions, colloidal suspensions, lotions, or any other form of carrier suitable to a particular mode and location of application. For example, the composition may contain one or more humectants, surfactants, preservatives, thickeners, fragrances, colorants, emmolients, and/or other suitable inactive ingredients.

More particularly, topical compositions useful in the subject invention may be formulated as a solution. Solutions typically include an aqueous solvent (e.g., from about 50% to about 99% or from about 90% to about 95% of a pharmaceutically acceptable aqueous solvent).

A solution of the invention may further comprise an emollient. Such compositions preferably contain from about 2% to about 50% of an emollient(s). As used herein, “emollients” refer to materials used for the prevention or relief of dryness, as well as for the protection of the skin. A wide variety of suitable emollients are known and may be used herein. See International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Ed., 1997) (hereinafter “CTFA Handbook”) contains numerous examples of suitable materials.

A lotion can also be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in the CTFA Handbook pp. 1693-1697.

The cobalamine compositions useful in the present invention may be formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, CTFA Handbook, pp. 1673-1686.

Lotions and creams can also be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion preparations, such as lotions and creams, of the oil-in-water type and water-in-oil type are well-known in the cosmetic art and are useful in the subject invention. Multiphase emulsion compositions, such as the water-in-oil-in-water type are also useful in the subject invention. In general, such single or multiphase emulsions contain water, emollients, and emulsifiers as essential ingredients.

The cobalamine actives and other compositions of this invention can also be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents.

Other inactive ingredients suitable for use in the invention generally include dimethicone, propylene glycol, propylene glycol stearate SE, glyceryl, polyglyceryl methacrylate, cetyl alcohol, triethanolamine, glyceryl stearate SE, octyl palmitate, tribehenin, sorbitan isostearate, potassium sorbate, cetyl palmitate, tristearin, quatemium-18 hectorite, bentonite, styearyl heptanoate, butyl stearate, palmitate, bisabolol, bis-diglyceryl polyacyladipate-2, polybutene, polysorbate-60, mica, ozokerite, phenyl trimethicone, diazolidinyl urea, imidazolidinyl urea, polymethacrylate, glycereth 26, biosaccharide gum, cetyl octanoate, sodium hyaluronate, polysorbate 20, peg-8/SMDI copolymer, palmitoyl-oligopeptide, disodium EDTA, trisodium EDTA, sodium ascorbyl phosphate, phenoxyethanol, suitable salts, benzyl nicotinate, pryidoxine, DL phenylannine, hexanicotinate, PVP/headecen copolymer, isopropyl palmitate, ceresin, PEG-100, stearate, phenylbenzimidazole, methyl gluceth 20, SD Alchohol 40-B, octyl palmitate, glycolic acid, polysorbate 80, ceteth-56, polyoxamer 401, octyl palmitate, silica, mica, fiber, phenoxyethanol, glyceryl polymethacrylate, stearic acid, citric acid, sodium lactate, cyclomethicone, cetyl octanoate, butylene glycol, squalane, ethylparben, phenoxyethanol, stearyl glycyrrhetinate, sodium glutamate, witch hazel, cellulose derivatives, tallow, lard, suet, butter, and wool fat.

Absorption promoters, detergents and chemical irritants (e.g., keritinolytic agents) can enhance transmission of a cobalamine composition across the dermis. Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in that text.

The cobalamine actives and compositions of the invention may also include additional co-active agents for treating or alleviating pain, such as pain reducers (including, without limitation, vitamin B-1, B-2, B-3, B-6, B-9, alpha-lipoic acid, the alpha or gamma linoleic and linolenic acids, and the amino acid derivative acetyl-1camitine), anti-inflammatories, analgesics and anesthetics. Those of ordinary skill in the art will be able to readily identify such agents for use in, in conjunction with, or apart from a cobalamine composition to be topically or transdermally delivered according to the invention.

Some amount of any of the inactive or co-active ingredients listed above may be present to suit a particular purpose and to provide a particular format for delivery of the cobalamine active; e.g., a lotion or gel. Thus, the pharmaceutically acceptable carrier component of a cobalamine composition of the invention may comprise from about 50% to about 99%, by weight, of the composition (e.g., from about 80% to about 95%, by weight, of the composition). For example, a mixture of about 50% cobalamine active w/w, with a balance of inactive ingredients, can generally be expected to produce good results. However, depending on the course of treatment being followed, cobalamine compositions of the invention can also be at least about 10%, about 20%, about 30%, about 40%, about 50%, about 60%, about 70%, about 80%, or about 90% cobalamine. It is most preferred that the cobalamine be present at less than 95% w/w and at more than 20%, 25%, or 35% ±5% (w/w). Other preferable amounts of the fatty acid ester include from about 40% to about 60% or from about 25% to 65% or between 45% and 55%, between 30% and 70%, between 35% and 65%, between 25% and 65%, or between 25% and 70% (w/w).

Further, the compositions of the invention include not only those in suitable form for direct topical or transdermal application but also concentrated primary compositions which can be supplied to the user for on-site dilution with a suitable quantity of water or other diluent before application. Typical examples are an aqueous solution, an aqueous dispersion or suspension, an aqueous emulsion, a concentrate emulsifiable in water or a dispersible powder. In such a concentrated primary composition or “concentrate”, the concentration of the active ingredient is brought to an effective level by addition of water or other diluting fluid, including for some applications a finely-divided powder.

A concentrate concentration of active can vary widely and can be for example from about 5% to about 50% or more of the concentrate, more preferably from about 5% to about 15%, to be diluted to a “working concentration” of less than about 10%, less than about 5%, less than about 4%, preferably less than about 3%, more preferably less than about 2%, more preferably less than about 1.5% or about 1%; or alternatively at a “working concentration” of at least about 0.25%, at least about 0.5%, more preferably at least about 0.75%, most preferably at least about 1%, at least about 1.5%, or at least about 2%. In a preferred embodiment, a preferred range of working concentration of cobalamine active is about 0.1% to about 5%, more preferably about 0.5% to about 2.5%, most preferably about 0.75% to 1.5%.

When prepared for administration (e.g., provided in a unit-dosage or reconstituted form), the cobalamine composition of the present invention has a pH greater that about 2 and a pH less than about 10 such as less than about 7, such as less than about 4.5.

C. Dosing Parameters.

1. Extended Delivery.

Those of ordinary skill in the clinical arts will be familiar with, or can readily ascertain, means for drug delivery allowing for release of a topically or transdermally delivered active over an extended period of time. A useful reference in this regard is Chien, Novel Drug Delivery Systems, Chapters 3 through 6 and 9 (Marcel Dekker, 1992), which chapters are incorporated herein.

For example, a colloidal dispersion system may be used for extended delivery of the cobalamine composition. Colloidal dispersion systems include macromole-cule complexes, nanocapsules, microspheres, beads, and lipid-based systems including oil-in-water emulsions, micelles, mixed micelles, and liposomes; however, in using lipid-based formulations, the lipid character of mucosae, which may be incompatible with lipid-based pharmaceutical delivery, must be taken into account. Phospholipid-based liposomes are easily prepared and commonly used, and may include phospolipids such as those composed of diester and triester phosphatides such as cocamidopropyl PG-dimonium chloride (Colalipid C™, Colonial Chemical, Inc., South Pittsburgh, Tenn., USA), stearamidopropyl PG-dimonium chloride (Colalipid ST™), sunfloweramidopropyl phosphate PG-dimonium chloride (Colalipid SUN™), Sodium olivamidopropyl PG-Dimonium Chloride Phosphate (Colalipid OL™), sodiumgrapeseedamindopropyl PG-dimonium chloride phosphate (Colalipid GS™), linoleamidopropyl PG-dimonium chloride phophate (Colalipid SAFEL™), PEG-8 dimethicone sunfloweramidopropyl PG-dimonium complex (Colalipid SIL™), ricinoleamidopropyl PG-dimonium chloride phosphate (Colalipid RC.™), sodium coco PG-dimonium chloride phosphate (Arlasilk™ phospholipids CDM, Uniqema, ICI Group of Companies, Wilton, UK), cocamidopropyl PG-dimonium chloride (Arlasilk™ phospholipids PTC), stearamidipropyl PG-dimonium chloride phosphate (Arlasilk™ phospholipids SV), linoleamidopropyl PG-dimonium chloride phosphate (Arlasilk™ phospholipids EFA), linoleamidopropyl PG-dimonium chloride phosphate dimethicone (Arlasilk™ phospholipids PLN), myristamidopropyl PG-dimonium chloride phosphate (Arlasilk™ phospholipids PTM), and sodium borageamidopropyl PG-dimonium chloride phosphate (Arlasilk™ phospholipids GLA), and combinations thereof.

Further, for transdermal delivery, transdermal patches may also be utilized for extended application of the cobalamine compositions of the invention. Other means for transdermal delivery of compounds are known in the art and suitable for use in the invention, such as iontophoresis, and sono-phonophoresis.

2. Suitable Dosages.

Cobalamine compositions of the invention and pain reducers, if utilized, may be administered according to the needs of the subject being treated and the dosage of active provided by the composition. In general, topically delivered dosages of at least about 50 kg/mg up to about 1000 mg/kg body weight can be expected to be suitable for use in the methods of the invention, with about 700 mg/kg being preferred.

Topical delivery of a cobalamine active and/or pain reducer may be achieved by application of a composition of the invention once a day, twice a day or more often. Transdermal application will be administered according to the requirements of the means utilized; e.g., a patch may be applied for a day, week or longer, while phoretic techniques may be utilized periodically as clinically indicated. It may be advisable to administer the cobalamine composition at first in a low dosage, then increase the dosage as needed to achieve the desired therapeutic goal. Based on current studies, vitamin B-12 and the preferred pain reducers mentioned herein (vitamin B-1, B-2, B-3, B-6, B-9, alpha-lipoic acid, the alpha or gamma linoleic and linolenic acids, and the amino acid derivative acetyl-1camitine) are not believed to exhibit any toxicity at these dosage levels.

C. Methods for Monitoring the Progression of Treatment According to the Invention.

Those of ordinary skill in the clinical arts will be familiar with methods for diagnosing peripheral neuropathy, and for determining the progression of the condition in response to treatment administered according to the invention.

For example, CTS is often determined by electrodiagnostic testing, with positive results correlated to clinical symptoms of pain, parasthesia, numbness and/or tingling along the path followed by the median nerve. Reduction in symptoms, especially pain, as by an order of at least one on a scale of 1-10 and/or by an improvement in the patient's global symptom score (GLSS) will be indicative of a therapeutic response.

Drug concentration in the tendon sheath tissue of the wrist obtained by biopsy may also be utilized to ensure entry into the subcutaneous tissue beneath the site of administration.

The invention having been fully described, its scope is defined by the appended claims.

I claim:

1. A method for alleviating pain associated with a peripheral neuropathy, the method comprising administering a pharmaceutically acceptable composition comprising a cobalamine active, wherein:
(a) the cobalamine is provided in a dosage sufficient to alleviate pain;
(b) the cobalamine composition is applied to skin at a site traversed or innervated by nerve affected by the neuropathy.

2. The method according to claim 1, wherein the cobalamine active is Vitamin B12.

3. The method according to claim 1, wherein the cobalamine composition consists essentially of Vitamin B12 and one or more of a pharmaceutically acceptable carrier, excipient, buffer or penetration enhancing agent.

4. The method according to claim 1, wherein the peripheral neuropathy is an focal peripheral neuropathy.

5. The method according to claim 4, wherein the focal peripheral neuropathy is carpal tunnel syndrome.

6. The method according to claim 5, wherein the cobalamine composition is applied to one or more of the wrists, forearm, elbow or shoulder to alleviate pain in the treated area.

7. The method according to claim 2, wherein the vitamin B12 is the sole pain alleviating agent in the composition.

8. The method according to claim 1, wherein the composition is topically deliverable.

9. The method according to claim 1, wherein the composition is transdermally deliverable.

10. The method according to claim 1, further comprising administering a pain reducer, anti-inflammatory or analgesic compound.

11. The method according to claim 10, further comprising administering a pain reducer, wherein the pain reducer is selected from the group of compounds consisting of vitamin B-1, B-2, B-3, B-6, B-9, alpha-lipoic acid, alpha linoleic acid, gamma linoleic acid, alpha linolenic acids, gamma linolenic acid, and acetyl-1camitine.
